(19) United States
(12) Patent Application Publication
(10) Pub. No.: US 2008/0188900 A1
(43) Pub. Date: Aug. 7, 2008

(54) HEART RATE REDUCTION METHOD AND SYSTEM

(75) Inventors: Howard Levin, Teaneck, NJ (US); Mark Gelfand, New York, NY (US)

Correspondence Address: NIXON & VANDERHYE, PC, 901 NORTH GLEBE ROAD, 11TH FLOOR, ARLINGTON, VA, 22203, US

(73) Assignee: G&L CONSULTING, LLC, New York, NY (US)

(21) Appl. No.: 11/962,966

(22) Filed: Dec. 21, 2007

Related U.S. Application Data
(60) Provisional application No. 60/871,229, filed on Dec. 21, 2006.

(51) Int. Cl. A61N 1/05

(52) U.S. Cl. 607/14

(57) ABSTRACT

A method and apparatus to slow a heart rate with subthreshold electric stimulation of the SA node. Stimulation is applied at a specific time in the cardiac cycle and at a specific subthreshold level. To control the heart rate, the stimulating signal may be modified automatically based on physiologic feedbacks. Stimulation may be applied using an implantable pulse generator directly to the SA node of the heart.

This application claims the benefit of the filing date of Dec. 21, 2006, of U.S. Provisional Patent Application Ser. No. 60/871,229, the entirety of which is incorporated by reference.

### BACKGROUND OF THE INVENTION

This invention relates to methods and apparatus for treatment of heart disease by reducing the patient's heart rate. It also relates to implantable electronic devices for subthreshold non-excitatory electric stimulation of the heart tissue and specifically of the SA node (sinoAtrial node) of the heart.

Rapid heart rates, or heart rates that are above the normal physiological range, are typically caused by an activation of compensatory physiologic mechanisms intended to increase oxygen delivery to the body during periods of increased metabolic demand, such as during exercise. While normal in many situations, in several chronic and acute heart disease states, the presence of a rapid heart rate is an index of the severity of the disease and may be deleterious in and of itself. A rapid heart rate can be a compensatory mechanism in diseases that cause hypotension (low blood pressure), leading to stroke, heart and kidney failure. Rapid heart rates can be present during panic attacks where while no physiological damage is done, the effects on the mental health and wellbeing of the person are devastating. Moreover, a rapid heart rate can itself cause damage such as following an acute myocardial infarction (MI) where a rapid heart rate can increase stress on the injured heart muscle and impede healing. Other situations where a rapid heart rate can have deleterious effects are in patients with coronary artery disease such as causing increased oxygen demand by the heart muscle, leading to ischemia and chest pain and leading to the development of systolic heart failure in patients with even normal hearts or worsening the function of the heart in patients with diastolic heart failure.

The proposed novel therapy can be useful in all of these clinical scenarios. Common to all of them: a) the heart rate is elevated above normal, b) it is desired to reduce it, and c) it is desired to reduce the heart rate without jeopardizing the needs of the body for oxygenated blood and/or causing other untoward physiologic responses of the body.

Congestive Heart Failure (CHF) is a major medical problem without a cure and is used to illustrate the preferred embodiment of the invention. It is understood the other cardiac conditions are equally important. CHF is a form of heart disease increasing in frequency. According to the American Heart Association, CHF is the “Disease of the Next Millennium.” The number of patients with CHF is expected to grow significantly as an increasing number of the “Baby Boomers” reach old age. CHF is a condition that can be associated with either a weakened heart that cannot pump enough blood to body organs (systolic heart failure) or a heart whose muscle is strong and can pump effectively but only pumps a reduced amount of blood primarily as a result of its inability to fully relax and properly fill with blood (diastolic heart failure).

In both the systolic (SHF) and/or diastolic (DHF) forms of CHF, the heart may be initially affected by to hypertension, vascular disease, valve disease and other conditions. CHF is a disease that typically worsens with time. Current treatments for CHF share the common goals of the alleviation of symptoms and the improvement of heart and kidney function. The cornerstone of the medical therapy of chronic systolic form of CHF (SHF) includes the use of angiotensin converting enzyme (ACE) inhibitors, positive inotropic agents, diuretics, and beta-blockers with the amount of each drug used dependent on the stage of heart failure. While drug therapy is effective in the early stages of CHF, there is no truly effective drug treatment for the later stages of CHF. Surgical solutions exist, but are only used for the treatment of very end-stage heart failure. These therapies (such as LVADs) are very effective at increasing blood flow. However, they are invasive, costly and require the patient to undergo heart transplantation.

While the prognosis of patients with DHF is a little better than for SHF, the complication rate is about the same as for SHF patients. DHF causes frequent outpatient visits and hospital admissions. The one year readmission rate is almost 50% in DHF patients.

The general approach to treating DHF is to reduce symptoms, mainly by lowering pulmonary (lung) pressures. The primary options for reducing lung pressures include reducing heart size, maintaining good pumping of the heart's upper chambers, and slowing the heart rate.

With some exceptions, many of the drugs used to treat systolic heart pressure are also used to treat diastolic heart failure. However, the reason they are used and the dose may be different for DHF. For example, while in SHF, beta-blockers are used to increase pumping power and reverse heart remodeling, in DHF, beta-blockers are used to make filling the heart with blood take longer, and to blunt the change in the heart's response to exercise. While diuretics are used in DHF, the diuretic dose is usually much smaller than for SHF. Calcium channel blockers have no place in SHF treatment but may help DHF.

However, a major clinical benefit is obtained by using therapies that slow the heart rate. This gives the heart more time to relax so it can fill with blood. Fast heart rates are poorly tolerated in DHF patients because rapid heart rate: (i) Increases the heart's oxygen demand and reduces blood flow to the heart, (ii) causes ischemia even without CAD Prevents full relaxation of the heart muscle, which raises pressure and reduces the heart's flexibility; (iii) Shortens the heart's relaxation period, making it incomplete, which reduces the amount of blood pumped per beat.

However, slowing the heart rate too much can reduce cardiac output despite better filling. This is why DHF patients need very individualized treatment. An initial goal might be a resting heart rate of about 60 beats per minute. As with any drug therapy, the ability to achieve the desired goals for clinical efficacy are complicated by the side effects and unpredictability of drug absorption, dosing and relative effects of the drug in an individual patients. Since heart rate reduction is a primary driver of clinical benefit in DHF, it would be very desirable to have a way of predictably, reversibly and accurately controlling heart rate in these patients.

Even with the wide variety of existing therapies, over 2,300,000 CHF patients become hospitalized each year at a cost of over $10 billion dollars to the health care system. New CHF therapies are clearly needed.

The body's vital organs (e.g., the brain, kidneys, liver, as well as the heart itself) require sufficient blood flow and pressure to allow normal homeostatic function. The relationship of blood flow to blood pressure in the body can be described by the following equation:

Blood Pressure=Blood Flow*Systemic Vascular Resistance.

In the normal resting patient, the heart pumps an average of 5 l/min of blood flow, termed cardiac output. The autonomic nervous system (as well as other homeostatic control systems) will increase or decrease arterial resistance as needed to maintain adequate perfusion pressure. Thus, for a constant blood flow, the higher systemic arterial resistance, the higher the blood pressure. Similarly, decreases in resistance with a constant blood flow will lower blood pressure. Another major mechanism to control vital organ perfusion is the ability of the heart to augment blood flow.

The amount of blood pumped by the heart, termed cardiac output, can be described by the following equation:

Cardiac Output (CO)=Heart Rate (HR)*Stroke Volume (SV).

SV is the amount of blood in milliliters pumped during one heartbeat. The heart can increase the amount of blood it pumps per heartbeat by increasing its force of contraction (termed contractility) or increasing preload, the volume in the ventricle at the end of diastole (termed end-diastolic volume or LVEDV). Thus, if systemic vascular resistance stays constant, increase stroke volume will increase the blood pressure, improving end-organ perfusion. Alternatively, increasing HR will also increase the cardiac output. In the normal situation, all of these mechanisms are active to various degrees in maintaining the optimal balance of blood pressure and flow.

CHF patients often have elevated heart rates, which may be considered a natural physiologic response to maintain cardiac output. In SHF, the SV is low because the heart muscle is weakened and has limited pumping capacity. In DHF, the pumping ability of the heart is normal but the filling volume of the heart is low as it can not relax properly. Hence, in either condition, an increase in heart rate is theoretically beneficial in increasing cardiac output.

However, it is know well known that an increase in heart rate may be detrimental to a diseased heart. In particular, the condition of a diseased heart typically worsens in response to an increase in heart rate. Thus, to improve the condition of CHF patients, most agree that the HR must be maintained as low as possible without jeopardizing the ability of the CHF patient to exercise reasonably without pain.

While there are known practical devices (pacemakers) that can be used to safely increase the heart rate, there are no clinically used devices capable of safely and reversibly reducing the heart rate. Heart rate can be reduced with drugs. Unfortunately drugs that reduce the heart rate also reduce contractility (strength of contraction) of the heart muscle. In CHF patients, it is desired to reduce the heart rate while maintaining or increasing contractility. For example, in the past two decades, the development of angiotensin converting enzyme (ACE) inhibitors and β-blockers has signaled perhaps the most significant development of this century in the pharmacological treatment of heart failure. Both are aimed at the neurohormonal axis of this disease and both act by disruption of the feedback loops that characterize heart failure. Both beta-blockers and ACE inhibitors are the first classes of drugs to be associated with a survival benefit for patients in heart failure. However, despite these significant advances in medical therapy, their effectiveness is limited, especially in the later stages of CHF. Patients become resistive to the increased dose and potency of drugs until further increase becomes too dangerous.

Beta-adrenergic blocker therapy reduces morbidity and mortality in patients with chronic heart failure (CHF). The role of beta-blocker-induced bradycardia (low heart rate) in improving left ventricular (LV) dysfunction in CHF has also been investigated. Prevention of tachycardia may contribute to clinical benefit after beta-blockade, as tachycardia can be both a cause and secondary effect of progressive CHF. Several studies suggest a role for bradycardia therapy in the treatment of CHF. Beta-blocker therapy, however, poses special challenges in the heart failure population, mainly due to its effects on systolic function. Certain populations of patients, such as those with reactive airway disease, conduction system disease, or hemodynamic compromise, may not tolerate therapy. This has lead to the need for dose titration and limitations on its clinical use. An ideal therapy for these patients can produce the same physiologic effects as a beta-blocker without the negative effects of beta-receptor blockade.

Electrical pulses in the heart are controlled by groups of cells with the special property of automaticity, or the ability to depolarize without external input or drive. The rhythm of the heart is normally determined by a pacemaker site called the sinoatrial (SA) node located in the posterior wall of the right atrium near the superior vena cava. The SA node consists of specialized cells that undergo spontaneous generation of action potentials at a rate of 100-110 action potentials (“beats”) per minute. This intrinsic rhythm is strongly influenced by autonomic nerves, with the vagus nerve being dominant over sympathetic influences at rest. This “vagal tone” brings the resting heart rate down to 60-80 beats/minute. The normal range for sinus rhythm is 60-100 beats/minute. Sinus rates below this range are termed sinus bradycardia and sinus rates above this range are termed sinus tachycardia.

The sinus rhythm normally controls both atrial and ventricular rhythm. Action potentials generated by the SA node spread throughout the atria, depolarizing this tissue and causing atrial contraction. The impulse then travels into the ventricles via the atrioventricular node (AV node). Specialized conduction pathways within the ventricle rapidly conduct the wave of depolarization throughout the ventricles to elicit ventricular contraction. Therefore, normal cardiac rhythm is controlled by the pacemaker activity of the SA node. Abnormal cardiac rhythms may occur when the SA node fails to function normally and other secondary pacemaker sites (e.g., ectopic pacemakers) trigger depolarization, or when normal conduction pathways are not followed.

As previously mentioned, the rate at which the SA node generated action potentials is under the influence of the autonomic nervous system. The sympathetic system innervates the heart and causes increase of the heart rate via B-1 adrenergic receptors, for instance as part of the “fight or flight” response. Conversely, the parasympathetic system, via the vagus nerve, slows the heart rate. If parasympathetic stimulation is increased, for instance by massaging the carotid sinus (baroreceptors), or by applying an electric field to the vagus nerve or its cardiac branches, the heart rate decreases. It is well known that electric stimulation of human parasympathetic efferent nerves that lie along the surface of the superior vena cava (SVC) or in the posteroinferior right atrium induces negative chronotropic and dromotropic effects. It also often causes other undesirable effects.

Electronic pacemakers can be used to replace or supplement the natural pacing nodes of the heart by applying electric excitory signals to the heart muscle to cause contraction and blood pumping cycle. Pacemakers are used in patients with diseased nodes (slow heart beat) and defective (blocked) conduction pathways.

Nerve stimulation has been proposed to treat cardiac disease but so far did not result in practical therapies because of the difficulty of applying stimulation to nerve fibers that are very small and fragile. Nerve bundles typically carry multiple signals (sensory and motor). This makes it very hard to achieve a desired specific and local effect of stimulation without also causing undesired effects. Many proposed inventions describe control of heart rate in patients by electric stimulation of parasympathetic nerves based on basic physiologic principles described above. None of them resulted in a practical therapy.

Contrary to cardiac pacemakers that apply electric stimulation to the heart muscle to cause contraction, Subthreshold Stimulation (STS) of the targeted cardiac tissue is non-excitatory stimulation and does not cause muscle fibers to contract. STS has been used or proposed to increase contractility of the heart muscle or to block electric conduction between the SA and AV node to reduce tachycardia (abnormally fast heart beat). It should be noted that nerve stimulation is different from the cardiac STS in that it applies electric stimulation to nerve fibers that innervate the heart and is therefore an indirect influence on heart activity.

### SUMMARY OF INVENTION

The invention breaks with tradition and proposes a counterintuitive novel method and apparatus of treating chronic CHF by controllably and reversibly slowing heart rate with subthreshold, non-excitatory stimulation of the SA node and surrounding heart muscle tissue. Application of electric stimulus to the SA node is generally avoided because, unlike the most of the cardiac muscle, the SA node does not have a substantial refractory period when it is insensitive to electric excitation. A significant property of the authors proposed method of subthreshold stimulation is that while the stimulus can trigger nerve activity, it does not trigger muscle (e.g., contractile) activity. The present invention uses subthreshold stimuli to affect the electrical activity of the SA node but is not intended to cause any clinically significant change in the contractile properties of the cardiac muscle.

To achieve the desired physiological and clinical effects, stimulation may be applied at a specific time in the cardiac cycle and at a specific subthreshold level. To control the heart rate, the stimulating signal may be modified automatically based on physiologic feedbacks by a control processor in an implanted pulse signal generator that senses signals of the cardiac cycle, such as electrical signals from the heart. A simple feedback is the heart rate that can be calculated from an endocardial electrogram based on an electrical sensor monitoring the heart. Stimulation is applied directly to the area of the SA node of the heart or the surrounding tissue using an implantable pulse generator.

The invention achieves its objective by pure modulation of the native pacemaker of the heart—SA node. In contrast, prior art attempted to indirectly affect SA node by manipulating parasympathetic innervations of the heart. The invention overcomes limitations of prior art by achieving the reduction of heart rate without the increase of the AV delay (long AV delay is generally not desired in heart failure patients) and depression of cardiac function. It also avoids other cardiac, pulmonary and gastric side effects of vagus nerve stimulation that so far prevented the adoption of vagus nerve stimulation for practical therapy of heart failure.

Inventors propose an implantable device and method for controllably slowing down the heart rate in a patient. The device (system) consists of an implantable pulse generator (IPG) with embedded intelligence such as a microprocessor and programmable logic capable of sensing heart electrical activity and administering electric stimulation to the SA node. The IPG is connected to the heart tissue by an implanted lead equipped with sensing and stimulating electrodes.

The invented therapy has negative chronotropic effect on the heart. Chronotropic effects are ones that change the heart rate (i.e. the time between p waves). The purpose of the invention is to stimulate postganglionic nerve terminals innervating the SA node without causing a heart contraction. While this action involves the nerve terminals of the parasympathetic nerves (e.g., vagus), it does so in a very local manner, does not directly stimulate or require other stimulation of the actual vagus nerve itself and thus avoids known complications and side effects associated with diffuse activation of vagus nerve stimulation. For that purpose, stimulation follows a known sub-threshold or non-excitory protocol. In one embodiment a pair of stimulation electrodes that can be separated by approximately 2 to 8 mm is placed directly on the endocardial surface of the SA node. Stimulation burst can be delivered for 100-200 ms and consists of 100-microseconds long rectangular pulses 2 to 15 V in amplitude and frequency of approximately 200 Hz, plus or minus 15 percent. The trains of pulses are triggered by the intracardiac electrogram and delivered sometime before and preceding the spontaneous action potential of the SA node or the heart ECG P-wave. The P-wave of the electrocardiogram (ECG) is an electrical signal of the heart having origins in the action of the SA node. These stimuli are expected to be subthreshold for sinoatrial or atrial muscle cells contraction but be of sufficient amplitude to stimulate postganglionic nerve terminals and delay the onset of the next P-wave thus making the heart cycle longer and the heart rate slower.

The invention consists of providing subthreshold, non-contractile stimuli applied to the directly to or to the area adjacent to the SA node. The SA node is clearly differentiated from the rest of the heart by its location, anatomic structure and function. The SA node consists of a cluster of specialized cells that have pacemaker activity (e.g., intrinsic automaticity). These cells are responsible for initiating the electrical impulse that stimulates the heart muscles to contract rhythmically. The where electric signals are applied to the SA node with implantable electrodes placed in close proximity to specialized SA node cells. The electric signals applied to the SA node are preferably non-destructive and are distinguishable from signals applied during ablation therapy to treat a diseases such as the “sick sinus syndrome” or “inappropriate tachycardia”. Application of the subthreshold signals locally and directly to the SA node should avoid side effects of vagus nerve stimulation, such as increased AV delay and reduction of heart muscle contractility.

In one embodiment of the present invention, a subthreshold stimuli is applied directly preceding normal initiation of the electric pulse by the functional SA node. In the preferred embodiment, the non-excitatory electric pulses are applied during the time window of 100 to 200 ms (milliseconds) before the P-wave on the surface ECG or the Right Atrial Action Potential on the atrial endocardial electrogram. To apply therapy, the P-wave of each heartbeat is anticipated using a predictive algorithm based on the previous heartbeats.

At this time there is no clear unifying scientific theory of why subthreshold stimulation of the SA node and/or adjacent areas delays the spontaneous action potential. One of multiple theories suggests that this stimulation causes the release of cholinergic neurotransmitters from the nerve terminals of the parasympathetic nerves specifically and locally innervating the SA node. Cholinergic means “related to the neurotransmitter acetylcholine”. A substance or stimulation is cholinergic if it is capable of producing, altering, or releasing acetylcholine. The parasympathetic nervous system is entirely cholinergic.

The SA node is made up of multiple different cells that create action potential at different rates. One set of cells has a baseline rate of generating action potentials at a rate of 100-110 per minute. Other SA node cells have intrinsic rates ranging from 60-90 impulses per minute. Generation of an action potential by one cell of the SA node resets all of the other cells in the SA node so the cells remain synchronized. Thus, if a cell with a higher intrinsic rate generates an impulse, the other cells with slower intrinsic rates are inhibited from generated their own impulse for that heartbeat.

These different cells in the SA node also are electrophysiologically heterogeneous with different sensitivity to cholinergic stimulation. Cholinergic stimulation leads to a shift in activity of the pacemaker from the cells with faster intrinsic rates to those with slower instrinsic rates. Thus, it can be speculated that subthreshold stimulation of SA node produces a region of transient increased cholinergic activity that slows the natural pacemaker rate of the heart.

### BRIEF DESCRIPTION OF THE DRAWINGS

A preferred embodiment and best mode of the invention is illustrated in the attached drawings that are described as follows:

FIG. 1 illustrates the invention.

FIG. 2 illustrates the alternative embodiment invention.

FIG. 3 illustrates the alternative embodiment invention.

FIG. 4 illustrates the timing of stimulation.

FIG. 5 illustrates the timing of a heart electric activity.

### DETAILED DESCRIPTION OF THE INVENTION

For the proposed clinical use, the capability of the invention is to controllably and reversibly reduce the heart rate with the goal of improving the patient's heart function and overall condition and ultimately to arrest or reverse the disease.

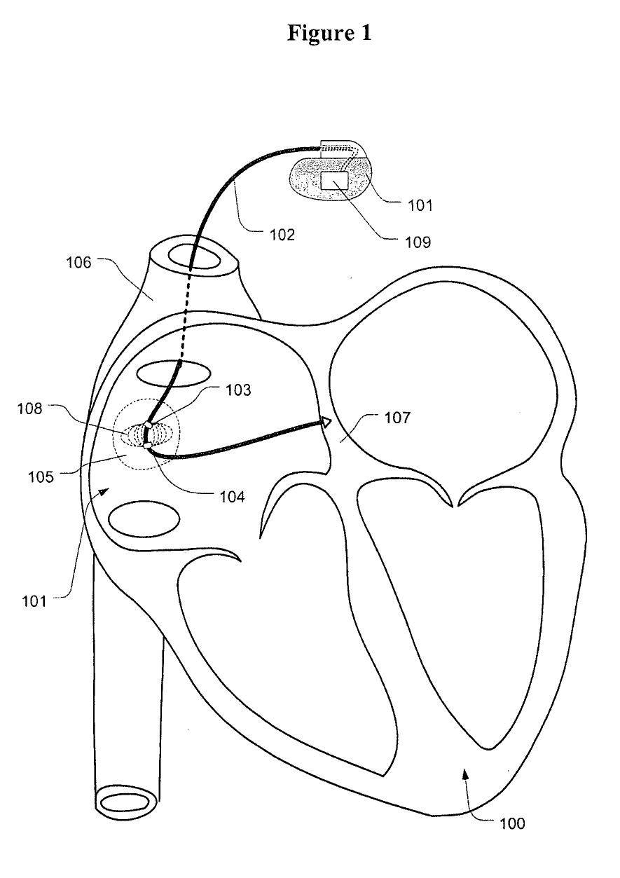

FIG. 1 illustrates the heart 100 treated with the invention. The IPG 101 is implanted in the patient's body using standard interventional cardiology techniques common to the implantation of pacemakers. The lead 102 is electrically connected to the IPG 101 and to the heart 100. It is understood that the IPG 101 can be also a cardiac pacemaker and can have more leads. It is expected that in future cardiac pacemakers will have ever more leads connecting them to various parts of the anatomy. The leads can combine sensing and pacing electrodes as known and common in the field. The IPG 101 is equipped with the embedded intelligence 109 that enables it to sense signals, process the information, execute algorithms and send out electric signals to the leads. The embedded intelligence such as a microprocessor with program and data memory and programmable logic capable of sensing heart electrical activity and administering electric stimulation is fairly common in the field of implantable electronic devices. It usually includes telemetry that allows programming and interrogation of the IPG. The IPG is equipped with a single use or rechargeable battery that supplies power for stimulation and intelligence.

In the disclosed embodiment, the distal end of the lead 102 resides in the right atrium of the heart 102. Lead 102 is equipped with electrodes 103 and 104 that are in the electric contact with the SA node 105. The SA node are impulse generating tissue in the right atrium of the heart and, particularly, is typically a group of cells positioned on the wall of the right atrium near the entrance of the superior vena cave. The lead 102 enters the right atrium 102 through the superior vena cava (SVC) 106 and is anchored in the atrial septum 107. Lead 102 can be introduced into the atrium of the heart straightened up by a removable stylet. After the distal tip of the lid is screwed into the heart muscle, the lead can be braced against the SA node to ensure tight electric contact with the SA node tissue. An electric field 108 is induced by the electric current applied by the positively charged anode and cathode lead electrodes. Electrodes are connected to the IPG 101 by wires that can be incorporated into the trunk of the lead 102. An electric field 108 is induced in the tissue of SA node to create temporarily desired local polarization that effects oscillatory pacemaker cells of the SA node situated in the close proximity of the electrodes 103 and 104.

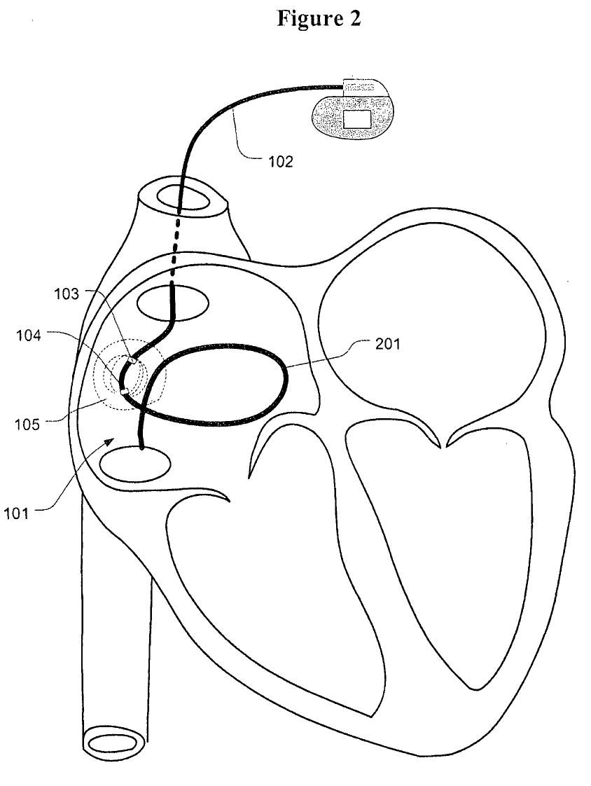

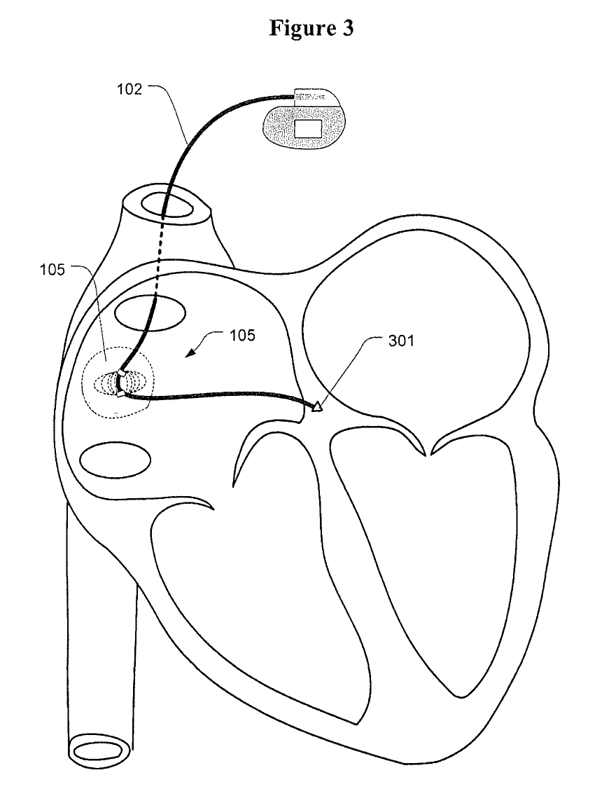

FIG. 2 illustrates an alternative embodiment of the lead 102. The lead, when inside the right atrium 101 forms a resilient loop 201 that braces against the walls of the atrium and presses electrodes 103 and 104 against the SA node tissue 105. FIG. 3 illustrates another alternative embodiment of the lead 102. The lead, when inside the right atrium 101 is anchored in the right atrial appendage (not shown) and braced against the SA node are 105. The lead is secured by the screw or barb tip 301 in the right atrial appendage. The tip of the lead can be equipped with additional electrodes for sensing and pacing of the heart.

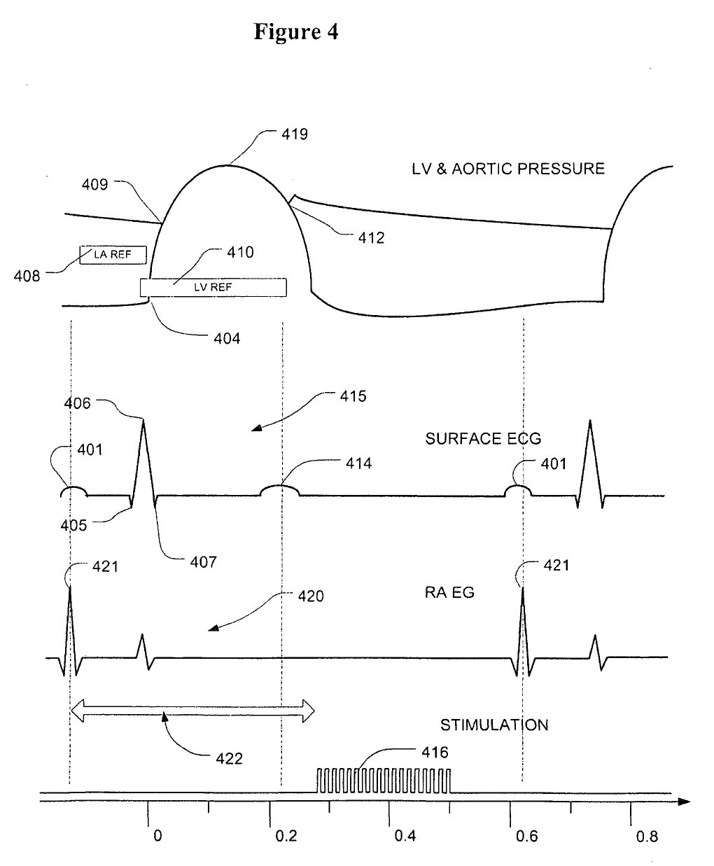

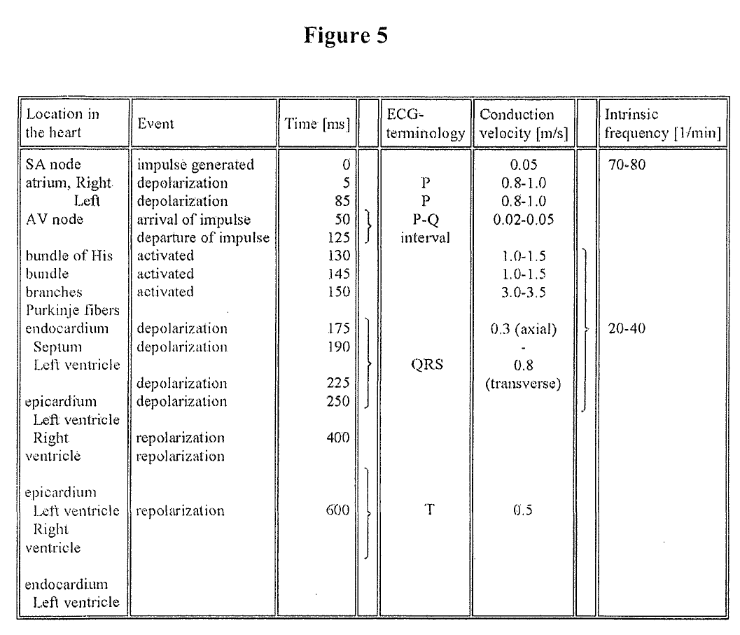

FIG. 4 illustrates stimulation of SA node with a sequence of stimulation pulses in relation to the timing of a heart cycle. Pulses are simplified and presented as a pulse burst 416 that comprise rectangular pulses spaced in time as represented by the X-axis that represents timing (in seconds) of a representative heart cycle. FIG. 5 is a table that provides further information about timing of electric activity events in the cardiac cycle.

The pulse burst 416 can, for example, comprise individual unipolar and/or biphasic (of alternating polarity) pulses. Pulse duration can be chosen from values between 0.05 to 0.15 milliseconds and delivered at frequency of 100 to 240 Hz, based on the existing general experience with nerve stimulation, to elicit chronoscopic effect in the SA node. In one preferred embodiment 0.1 ms long bipolar pulses are delivered for 100 ms at 200 Hz, at the amplitude of 10 V. It is preferred to apply pulses of lowest possible amplitude and duration that will ensure the desired response without causing undesired activation of electrical or mechanical activity of the tissues.

As previously noted, the amount of energy required to cause these undesired stimulations varies depending if the tissues in contact with the electrodes. It may be desired to alter the stimulation pattern during the pulse burst such that the energy delivered is sufficient to delay the P-wave of the next heart beat just the desired amount for the particular patient. Since the patients ECG or electrogram is constantly monitored by the device, the parameters of stimulation can be altered by changing either the pulse duration, pulse amplitude or both. The duration of the pulse burst can be automatically determined, such using a percentage of the period between heart beats, or user-set.

Based on the existing experience, pulses in the range of 2 to 20 Volts (V) and preferably less than 10 V should be sufficient to subthreshold stimulate SA node if the electrodes are in a good contact with the SA tissue. It is desired to maintain amplitude below the level that can cause irregular heart beats (arrhythmias), inadvertent heart muscle contraction, skeletal muscle twitching and pain. It is possible to include means to adjust these parameters after the implantation, using the stimulator's telemetry capability embedded in the IPG logic. The amplitude and frequency of the STS may vary burst to burst or pulse by pulse—within the same burst of pulses—for a single burst waveform. The burst duration can be in the range of 0.1 to 0.25 seconds, the ultimate limiting factor being the duration of the T-P period of the heart.

The IPG intelligence, e.g., a microprocessor 109 housed in the implant 101 (See FIG. 1), may adjust the stimulation burst shape, pulse shape, frequency of pulses and amplitude of pulses to set or control the blood pressure. The system may also adjust the rate of rise and fall of the pulse amplitude within the burst to create ramps of variable shape. The microprocessor or monitors the heart, such as by sensing electric signals from the heart, e.g., ECG signals, pressure signals from a pressure sensor or oxygen saturation signals from an oxygen saturation sensor in the heart or vascular system. The microprocessor executes an algorithm that determines the burst shape, pulse shape, frequency of pulses and/or amplitude of pulses based on the sensor input signals.

FIG. 4 also illustrates the concept of the heart cycle synchronized subthreshold stimulation. The heart (See FIG. 1) has intact electric conduction including a substantially normal physiologic A-V node conduction delay as further illustrated by the timing table on FIG. 5. Stimulation in this embodiment is implemented by electric stimulation with epicardial electrodes (See FIGS. 1, 2 and 3). Sensing of the cardiac electric activity can be performed with the same leads or additional atrial or ventricular leads known in the field of pacemakers.

The natural pacemaker or SA node of the heart initiates the heart cycle with the P wave 401 of the ECG that corresponds to the beginning of atrial contraction. The surface ECG P-wave corresponds to the right atrial muscle action potential 421 on the RA endocardial electrogram 420. It is also the beginning of the heart systole. During atrial contraction, atrial pressure increases and atrial volume decreases. The end of this time period corresponds to the beginning of the atrial refractory period 408. During this period, the atria can not be paced to contract. The P wave 401 of the surface ECG is followed by the Q wave 405 that signifies the beginning of the isovolumic contraction of the ventricle. Ventricular pressure 404 rise begins rapidly. In response the Tricuspid and Mitral valves of the heart close. Ventricular refractory period 410 begins. At the end of isovolumic contraction 409 The Pulmonary and Aortic valves open and the ejection of blood from the ventricle begins. Ventricular pressure reaches its peak in the middle of systole 419. The atrium is passively filled with blood as it relaxes. Approximately by the middle of systole both heart atria are filled with blood and their refractory period 408 is over. Atria are primed for a new contraction while the ventricle is ejecting blood. A-V valves are closed. At the same time the ventricle is still refractory and will not start another contraction in response to a natural or artificial pacing stimulus. Heart waves Q 405, R 406 and S 407 are commonly used markers of the beginning of the isovolumic contraction and the beginning of ventricular ejection (S wave). All modern pacemakers are equipped with means to read and analyze the endocardial electrogram such as the atrial electrogram illustrated by the trace 420 that are suitable for this embodiment of the invention.

Systole ends when the aortic valve closes 412. Isovolumic relaxation of the ventricle starts. This point also corresponds to the middle of the T wave 414 of the ECG. Importantly for the invention, the middle of T wave 414 corresponds to the end of the absolute refractory period 410 of the ventricle. At the end of the T-wave, the Tricuspid and Mitral valves open and the atrium volume starts to drop as the blood starts to flow from the atria into ventricles to prime them for the next ventricular contraction and ejection.

For this embodiment, the stimulation burst 416 starts after the end of the calculated delay 422 that can be approximately 200 ms after the P-wave or monitored action potential 421. The stimulation burst 416 can be repeatedly applied over sequential or spaces out heartbeats for the duration of therapy. It is possible that some patients will not need or will not be able to tolerate continuous stimulation 24 hours a day. In such patients period of normal heart activity can be followed by the period of stimulation followed again by the rest period. Switching between stimulated and natural modes can be based on timing, patient's activity or physiologic feedbacks.

The invention has been described in connection with the best mode now known to the applicant inventors. The invention is not to be limited to the disclosed embodiment. Rather, the invention covers all of various modifications and equivalent arrangements included within the spirit and scope of the appended claims. Common to all the embodiments, is that the implantable device is used to exert affect on the SA node locally by electrically stimulating it below the level of cardiac contractions. The invention has been described in connection with the best mode now known to the applicant inventors. The invention is not to be limited to the disclosed embodiment. Rather, the invention covers all of various modifications and equivalent arrangements included within the spirit and scope of the appended claims.

While the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the appended claims.

What is claimed is:

1. A method to reduce a heart rate comprising:
slowing the heart rate of a patient by applying an artificial subthreshold, non-excitatory stimulation of a sinoAtrial node (SA node) of a heart in the patient.

2. The method of claim 1 wherein the subthreshold, non excitatory stimulation does not trigger muscle contractile activity.

3. The method of claim 1 wherein the stimulation is generated by an implanted pulse generator and applied by an electrode implanted in the patient proximate to the SA node.

4. The method of claim 3 wherein the electrode includes a pair of stimulation electrodes separated by between 2 to 8 mm and placed on an endocardial surface of a SA node of the heart.

5. The method of claim 1 wherein the stimulation includes a stimulation burst having a period of 100 millisecond to 200 milliseconds.

6. The method of claim 5 wherein the burst is a plurality of bursts having a frequency of approximately 200 Hertz.

7. The method of claim 5 wherein the stimulation burst is delivered before a heart ECG P-wave.

8. A method to reduce a rate of beating in a heart of a human patient comprising:
implanting at least one stimulation electrode proximal to a sinoAtrial node (SA node) of the heart;
implanting an pulse generator in the patient, and
applying a non-excitatory electric stimulation to the SA node, wherein the stimulation is generated in the pulse generator and applied by the stimulation electrode.

9. The method of claim 8 wherein the non-excitatory stimulation stimulates the SA node to delay a subsequent P-wave of the heart.

10. The method of claim 8 wherein the stimulation is applied at a voltage level in a range of two volts to twenty volts.

11. The method of claim 8 wherein the non-excitatory stimulation is subthreshold stimulation (STS).

12. The method of claim 8 wherein the non-excitatory stimulation avoids causing contraction of muscle fibers in the heart.

13. The method of claim 8 wherein pulse generator is a modified pacemaker.

14. An apparatus to reduce a heart rate in a human patient comprising:
a stimulation electrode adapted to be implanted proximal to a sinoAtrial node of a heart in a human patient;
a pulse generator in communication with the stimulation electrode, wherein the pulse generator receives a signal indicative of a condition of an electrical cycle of the heart and generates a subthreshold stimulation signal applied by the electrode to the sinoAtrial node.

15. The apparatus of claim 14 wherein the pulse generator is an implantable pulse generator and the apparatus includes a wire lead between the generator and electrode to provide the communication.

16. The apparatus of claim 14 wherein the stimulation signal has a potential in a range of 2 to 20 volts, and consists of signal bursts each having a during of less than 100 microseconds.

17. The apparatus of claim 14 wherein the signal bursts have a frequency of approximately 200 Hertz.

18. The apparatus of claim 14 wherein the stimulation electrode includes a pair of electrodes and separated by between two to eight millimeters when applied proximate to the SA node.

19. The apparatus of claim 14 wherein the stimulation is a signal is applied preceding normal initiation of an electric pulse by the SA node, wherein the pulse generator determines the normal initiation based on a sensor monitoring an electrical cycle of the heart.

20. The apparatus of claim 19 wherein the stimulation signal is initiated 100 to 200 milliseconds before an expected P-wave of the electrocardiogram of the heart.
